(19) United States
(12) Patent Application Publication
(10) Pub. No.: US 2015/0099301 A1
(43) Pub. Date: Apr. 9, 2015

(54) CELL CULTURE FLUID

(71) Applicant: Konkuk University Industrial Cooperation Corp., Seoul (KR)

(72) Inventors: Jeong Ik Lee, Seoul (KR); Soo Jung Lee, Seoul (KR); Kyung Mi Lee, Seoul (KR)

(21) Appl. No.: 14/381,417

(22) PCT Filed: Feb. 28, 2013
(86) PCT No.: PCT/KR2013/001675

§ 371 Date: Aug. 27, 2014

(30) Foreign Application Priority Data
Feb. 28, 2012 (KR) 10-2012-0020505
Feb. 27, 2013 (KR) 10-2013-0021590

(51) Int. Cl. C12N 5/00

(52) U.S. Cl. CPC C12N 5/0018; C12N 2500/38; C12N 2500/05; C12N 2500/32
USPC 435/408; 435/404

(57) ABSTRACT

The present invention relates to a cell culture medium and more particularly, to a safe, stable, and effective cell culture medium for cell therapy products.

### TECHNICAL FIELD

The present invention relates to a cell culture medium and more particularly, to a safe, stable, and effective cell culture medium for cell therapy products.

The present invention is supported by the Health and Medical Research and Development Project of the Ministry of Health and Welfare (A120275).

### BACKGROUND ART

Since animal cells were first cultured (1907), there has been no change in composition of culture media currently available on the market. After development carried out mainly from the late 1950s to 1960s, there has been little research on composition of culture media.

Further, as a cell culture medium for cell therapy products for culturing human cells in vitro for a certain period of time and returning the human cells to a human body, culture media available on the market have been used in most cases.

Most of the culture media currently available on the market contain a pH indicator and a substance for regulation (phenol red, HEPES). Some of these substances have not yet been proven safe regarding carcinogenesis, malformation, and development disorder when exposed to a human body for a long time, and, thus, they can be potential carcinogens, toxic substances, and the like and there are hazardous substances which cannot be directly used to a human body. Further, in order to promote cell proliferation, the culture media currently available on the market usually contain heterogeneous animal-derived (Xeno) components such as fetal bovine serum (FBS), and, thus, there is a risk of infection of a heterologous infectious disease in a culture stage. Therefore, safety of a cellular material of a vivo-derived cell therapy product cannot be secured.

Accordingly, if a culture medium having a composition capable of culturing cells is developed by investigating various already approved infusion preparations and injection preparations currently used in clinical trials for treating diseases in hospitals, it is possible to develop a culture medium capable of ensuring safety of a culture process of a cell therapy product.

As a relevant prior patent, Korean Patent Laid-open Publication No. 1020060052158 relates to “Cell preservation liquid”, and has an object to provide a cell preservation liquid capable of cryopreservation and cold preservation of cells with improved cell-preservation ability, and a simple method of preserving cells using the preservation liquid. It is described that the above-described object can be realized by a cell preservation liquid which contains at least saccharide, sodium ions, and potassium ions and in which a molar concentration of the sodium ions is equal to or higher than a molar concentration of the potassium ions, and, thus, cells can be cryopreserved and cold-preserved and a simple method of preserving cells can be provided, and also, the cell preservation liquid of the present invention makes it simple to culture cells since it is possible to culture cells by inoculating the cells in a suspended state in the preservation liquid to a medium without a process such as washing.

As another relevant prior patent, Korean Patent Laid-open Publication No. 1020070122316 relates to “Soft tissue filler composition for injection and preparation method thereof” and is about a soft tissue filler composition for injection and a preparation method thereof and more particularly, to a preparation method of a soft tissue filler composition for injection including: 1) digesting an autologous dermal tissue isolated from a patient's own skin and separating into single cells; 2) culturing and proliferating the isolated dermal cells via in vitro serum-free cultivation to obtain a cell culture medium containing dermal fibroblast stem cells, dermal fibroblast transit-amplifying cells, dermal fibroblasts and/or collagen protein; 3) centrifuging the cell culture medium to obtain a cell deposit; and 4) suspending the cell deposit in a glucose injection solution or a random injection solution to prepare a suspension for injection; and a soft tissue filler composition for injection prepared by the same. It is described that the soft tissue filler composition prepared by the method of the present invention exerts prolonged therapeutic effects without causing any immune response and side-effect due to the use of autologous dermal cells, and since it rules out the risk of the use of animal-derived serum through serum-free cultivation and immediately exhibits therapeutic effects by producing a large quantity of collagen protein, the soft tissue filler composition can be effectively used for improving skin tone and resilience as well as smoothing and removing wrinkles and scars.

### DISCLOSURE

### Technical Problem

The present invention is conceived by the aforementioned need to solve the above problems, and an object of the present invention is to provide a safe, stable, and effective cell culture medium.

### Technical Solution

In order to achieve the above object, the present invention provides a preparation method of a cell culture medium, including: preparing a culture medium having a composition similar to a cell culture medium available on the market by mixing an infusion or injection preparation for amino acid supply, an infusion or injection preparation for vitamin supply, an infusion or injection preparation for inorganic salt supply, an infusion or injection preparation for glucose supply, other supplement infusion or injection preparations, and an antibiotic and antimycotic infusion or injection preparation.

In an exemplary embodiment of the present invention, the infusion or injection preparation for vitamin supply may be one or a combination selected from the group consisting of Tamipool inj. (Celltrion Pharma. Inc.), Cetnevit inj. (Baxter), INFUVITE ADULT (Sandoz Canada Inc.), Infuvite (Sandoz Canada Inc.), M.V.I. ADULT™ (Hospira Worldwide, Inc.), M.V.I. inj. (SS), M.V.I. kit, M.V.I.-9 inj. (SS), M.V.I.-3 inj. (SS), M.V.I.-12 kit (SS), Otsuka MV inj. (Otsuka), and Sorvita (Huso), but may not be limited thereto.

In another exemplary embodiment of the present invention, preferably, the infusion or injection preparation for amino acid supply may be a preparation selected from the group consisting of Clinimix 85 inj. (Ilyang Pharmaceutical Co., Ltd.), Choongwae Tropamin Injection 6% (Choongwae Pharma, Co., Ltd.), Choongwae Hepatamine Injection (Choongwae Pharma, Co., Ltd.), Cafsol Injection (Chong Kun Dang Pharmaceutical Corp.), Yungjin Intrafusin 10% Injection (Yungjin Pharmaceutical Co., Ltd.), Proamin Injection 10% (Hanall Biopharma), Hepavia Injection (Dai Han Pharm. Co., Ltd.), Liversol Injection (Shin Poong Pharm. Co., Ltd.), Hanall Morihepamin Injection (Hanall Biopharma), Nephrisol 5.6% (Dai Han Pharm. Co., Ltd.), Glamin Injection (Fresenius Kabi Korea), Primene 10% (Baxter), Doctorlamine Injection (Choongwae Pharma, Co., Ltd.), Aminoglu Injection (MG Medi Green), Aminoven Infant 6% Injection (Fresenius Kabi Korea), Aminosteril-KE Injection 10% (Dai Han Pharm. Co., Ltd.), Aminofusin-TPN Injection (Yungjin Pharmaceutical Co., Ltd.), Combiflex MCT Plus Injection (Choongwae Pharma, Co., Ltd.), Combiflex MCT Peri Injection (Choongwae Pharma, Co., Ltd.), Combiflex MCT Special Injection (Choongwae Pharma, Co., Ltd.), Saeronamin Injection (Dai Han Pharm. Co., Ltd.), 5.4% NephrAmine Injection (B. Braun Medical Inc.), Aminosyn II Injection (Hospira, Inc.), Aminosyn Sulfite-Free (Hospira, Inc.), HepatAmine Injection (B. Braun Medical Inc.), TrophAmine Injection (B. Braun Medical Inc.), AMINOSYN II 3.5% in 25% Dextrose Injection (Hospira, Inc.), 10% Travasol Injection (Baxter Healthcare Corporation), Amicaliq (Terumo) Amizet XB (Terumo), Amino Furit, Aminic, Amiparen, ES-Polytamin, Istr, Moriamin S, Ispor S 12%, Amin 12 Proteamin 12, Amin 12 X Proteamin 12 X, Amiyu, Plus Amino, Macamine, and Moripron F, but may not be limited thereto.

In yet another exemplary embodiment of the present invention, preferably, the infusion or injection preparation for inorganic salt supply may be one or a combination selected from the group consisting of Daihan Hartmann Glucose Injection (Dai Han Pharm. Co., Ltd.), Hartmann Solution (Dai Han Pharm. Co., Ltd.), Hartmandex Solution (Choongwae Pharma, Co., Ltd.), Hartman Solution (Choongwae Pharma, Co., Ltd.), CJ Hartman Solution (CJ Cheiljedang), CJ Hartman-D Solution (CJ Cheiljedang), B.Braun Hartmanns Injection (B. Braun Korea), Daihan Isotonic Sodium Chloride Injection, Lactated Ringer's Injection (Medex Supply), Braun Lactated Ringer's Injection (Allegro Medical), Plasma-Lyte M and 5% Dextrose Injection, Lactated Ringer and 5% Dextrose Injection, EL-3 (Ajinomoto Pharmaceutical Co., Ltd.), Lactate Ringer's Solution, Ringer's Solution (I'Rom Pharmaceutical Co., Ltd.), Solita Injection (Ajinomoto Pharmaceutical Co., Ltd.), Solita-T1 Injection (Ajinomoto Pharmaceutical Co., Ltd.), Solita-T2 Injection (Ajinomoto Pharmaceutical Co., Ltd.), Solita-T3 Injection (Ajinomoto Pharmaceutical Co., Ltd.), Solita-T4 Injection (Ajinomoto Pharmaceutical Co., Ltd.), and Hartmann's Solution “Kobayashi” 500 mL calcium chloride dihydrate, but may not be limited thereto.

Further, preferably, the preparation method of a cell culture medium may further comprise adding an infusion or injection preparation for glucose supply, other supplement infusion or injection preparations, and an antibiotic and antimycotic infusion or injection preparation, but may not be limited thereto.

Furthermore, the present invention provides a cell culture medium composition including: an infusion or injection preparation for vitamin supply; an infusion or injection preparation for amino acid supply; and an infusion or injection preparation for inorganic salt supply.

In still another exemplary embodiment of the present invention, preferably, the composition may be used as a general conservative solution or an immersion solution for cells and tissue for a short time or for a long time, or a portable solution for easy delivery, but may not be limited thereto.

In still another exemplary embodiment of the present invention, preferably, the composition may be used as a component of a preservation solution for preservation and delivery of an organ to be transplanted into a body, but may not be limited thereto.

In still another exemplary embodiment of the present invention, preferably, the composition may be used as an alternative to a buffer solution used in a molecular biological or biochemical experimental protocol, but may not be limited thereto.

Further, the present invention provides a preparation method of a micro bubble solution or a nano bubble solution using the composition for preparing a micro bubble solution or a nano bubble solution by cutting an oxygen, carbon dioxide, ozone, nitrogen, hydrogen, or hydrogen sulfide gas, which has a characteristic usable for a certain use or purpose, into micro- or nano-sized bubbles in a solution.

Furthermore, the present invention provides a preparation method of a complete medium for cell therapy products, including: adding serum derived from cord blood or autologous blood to the composition of the present invention.

Moreover, the present invention provides a complete medium composition for cell therapy products, including: the composition of the present invention; and serum derived from cord blood or autologous blood.

Besides, the present invention provides a cell culture medium composition which is prepared by using a conditioned medium obtained after a target cell is cultured with the cell culture medium composition of the present invention or performing freeze-drying and sterilization to the conditioned medium so as to be a material in powder form, wherein these materials can be used for treatment, regeneration, health improvement through a transfer into a body including injection.

A list of medicines used as components of fluid-based culture medium (FCM) in the present invention is as follows.

For supply of inorganic salts, the following base solution products described in 1), for supplement, inorganic salt components described in 2) to 5) may be used.

1) Base Solution

A colorless and transparent solution in a polypropylene, polyvinyl chloride, or glass container

1)-1 Daihan Hartmann Glucose Injection 500 ml, 1000 ml (Dai Han Pharm. Co., Ltd.)

Calcium Chloride 20 mg, Dextrose 5 g, Potassium Chloride 30 mg, Sodium Chloride 600 mg, and Sodium Lactate 620 mg per 100 ml

1)-2 Hartmann Solution 500 ml, 1000 ml (Dai Han Pharm. Co., Ltd.)

Calcium Chloride 0.27 g, Potassium Chloride 0.4 g, Sodium Chloride 6 g, and Sodium Lactate 3.22 g per 1 L

1)-3 Hartmandex Solution (Choongwae Pharma, Co., Ltd.)

Calcium Chloride 20 mg, Potassium Chloride 30 mg, Sodium Lactate 620 mg, Dextrose 5 g, and Sodium Chloride 600 mg per 100 ml

1)-4 Hartman Solution (Choongwae Pharma, Co., Ltd.)

Calcium Chloride 0.27 g, Potassium Chloride 0.4 g, Sodium Chloride 6 g, and Sodium Lactate 3.22 g per 1 L

1)-5 CJ Hartman Solution 500 ml, 1000 ml (CJ Cheiljedang)

Calcium Chloride 0.27 g, Potassium Chloride 0.4 g, Sodium Chloride 6 g, and Sodium Lactate 3.22 g per 1 L

1)-6 CJ Hartman-D Solution 500 ml, 1000 ml (CJ Cheiljedang)

Calcium Chloride 20 mg, Dextrose 5 g, Potassium Chloride 30 mg, Sodium Chloride 600 mg, and Sodium Lactate 620 mg per 100 ml

1)-7 B.Braun Hartmanns Injection 500 ml, 1000 ml (B. Braun Korea)

Calcium Chloride 0.027 g, Potassium 0.04 g, Sodium Chloride 0.6 g, and Sodium Lactate 0.312 g per 100 ml

1)-8 Daihan Isotonic Sodium Chloride Injection 50 ml, 100 ml, 150 ml, 200 ml, 500 ml, 1000 ml, 3000 ml

Sodium Chloride 2.25 g per 250 ml

2) MgSO₄ anhyd.

A colorless and transparent glass ampoule filled with a colorless and transparent or an aqueous injection container made of plastic

2)-1 Daihan Magnesium Sulfate Injection 10%, 50% (Dai Han Pharm. Co., Ltd.)

Magnesium Sulfate 2 g per 20 ml/A

2)-2 Magnesin Injection (Daewon Pharm. Co., Ltd.)

Magnesium Sulfate 10 g per 20 ml/A

2)-3 Jeil Magnesium Sulfate Injection 50% (Je Il Pharmaceutical Co., Ltd.)

Magnesium Sulfate 10 g per 20 ml/A

2)-4 Masi Injection 10%, 50% (Huons Co., Ltd.)

Magnesium Sulfate 10 g per 20 ml/A

3) Fe(NO₃)₃.9H₂O

An injection of a dark brown opaque aqueous solution in a colorless and transparent vial

3)-1 Ferinject Injection 2 ml, 10 ml

Ferric hydroxide carboxymaltose complex 180 mg (50 mg as Fe) per 1 ml

3)-2 Venoferrum Injection (Choongwae Pharma, Co., Ltd.)

Ferric hydroxide sucrose complex 2700 mg (20 mg/ml as Fe) per 5 ml

3)-3 Ferrum Mate Solution (Choongwae Pharma, Co., Ltd.)

Ferric hydroxide Polymaltose complex 357 mg (100 mg as Fe3+) per 1 ml/A and 5 ml

3)-4 Ferrumkid Solution (Choongwae Pharma, Co., Ltd.)

Ferric hydroxide Polymaltose complex 17.85 g (5 g as Fe3+) per 1 ml/A and 100 ml

3)-5 Ferrum I Solution (JW Shinyak)

Ferric hydroxide Polymaltose complex 17.85 g per 100 ml

4) NaHCO₃

A colorless liquid in a colorless ampoule or a colorless liquid in a colorless and transparent plastic (polypropylene, polyethylene) container

4)-1 DHDEX 0.15% Solution 2 (Dai Han Pharm. Co., Ltd.) 12.6 L/bag, Sodium Bicarbonate 5.83 g

4)-2 Fresol (Part B) Solution 10 L, 12.6 L (Dai Han Pharm. Co., Ltd.)

10 L/bag, Sodium Bicarbonate 840 g per 10 L

12.6 L/bag, Sodium Bicarbonate 1058.4 g per 12.6 L

4)-3 Choongwae Sodium Bicarbonate Injection 8.4% (Choongwae Pharma, Co., Ltd.)

20 ml/A, Sodium Bicarbonate 1.68 g

4)-4 Huons Sodium Bicarbonate Injection 8.4% (Huons Co., Ltd.)

20 ml/A, Sodium Bicarbonate 1.68 g

4)-5 Daewon Sodium Bicarbonate Injection 8.4% (Daewon Pharm. Co., Ltd.)

20 ml/A, Sodium Bicarbonate 1.68 g

4)-6 Jeil Sodium Bicarbonate Injection 8.4% (Je Il Pharmaceutical Co., Ltd.)

20 ml/A, Sodium Bicarbonate 1.68 g

5) NaH₂PO₄, Na₂HPO₄

A colorless and transparent liquid medicine

5)-1 Leclean Solution (Dongindang Pharmaceutical Co., Ltd.)

1 ml/A, Dibasic sodium phosphate 60 mg, Monobasic sodium phosphate 160 mg

5)-2 Solin Enema (Korea Pharma Co., Ltd.)

1 ml/A, Dibasic sodium phosphate 60 mg, Monobasic sodium phosphate 160 mg

5)-3 Fleet Phosphosoda (Unimed Pharm Inc.)

1 ml/A, Dibasic sodium phosphate 180 mg, Monobasic sodium phosphate 480 mg

5)-4 Fleet Enema (Unimed Pharm Inc.)

1 ml/A, Dibasic sodium phosphate 60 mg, Monobasic sodium phosphate 160 mg

5)-5 Colclean Solution (Taejoon Pharm Co., Ltd.)

1 ml/A, Dibasic sodium phosphate 180 mg, Monobasic sodium phosphate 480 mg

5)-6 Colclean-S Solution (Taejoon Pharm Co., Ltd.)

1 ml/A, Dibasic sodium phosphate 60 mg, Monobasic sodium phosphate 160 mg

5)-7 Tamezon Injection (Kukje Pharma)

1 ml/A, Betamethasone sodium phosphate 5.2 mg (4 mg/ml as Betamethasone)

5)-8 Hanall Betamethasone Injection (Hanall Biopharma)

1 ml/A, Betamethasone sodium phosphate 5.2 mg (4 mg/ml as Betamethasone)

5)-9 Medica Betamethasone Sodium Phosphate Injection (Medica Korea)

1 ml/A, Betamethasone sodium phosphate 5.2 mg (4 mg/ml as Betamethasone)

5)-10 Dongkwang Betasone Injection (Dongkwang Pharm. Co., Ltd.)

1 ml/A, Betamethasone sodium phosphate 5.2 mg (4 mg/ml as Betamethasone)

As the other components,

1) Glucose may include one selected from the following products.

A colorless liquid in a colorless ampoule or a colorless liquid in a colorless and transparent plastic (polypropylene, polyethylene) container

1)-1 Samsung Glucose Injection 5% (Samsung Pharm. Ind. Co., Ltd.)

Glucose N/A

1)-2 Choongwae Dextrose Injection 50% 100 ml, 500 ml

Dextrose 50 g per 100 ml

1)-3 Marodex-D Injection (Ilsung Pharmaceuticals Co., Ltd.)

Dextran N/A, Glucose N/A

1)-4 Kwangmyung Dextrose Injection (Huons Co., Ltd.)

Glucose N/A

1)-5 Micicola Solution 50, 75, 100 (Dongindang Pharmaceutical Co., Ltd.)

Glucose 50 g, 75 g, 100 g

1)-6 Diasol-S Solution (Taejoon Pharm Co., Ltd.)

Glucose 0.5 g per 1 ml

1)-7 Hemadex-D Injection (Samsung Pharm. Ind. Co., Ltd.)

Dextran N/A, Glucose N/A

1)-8 Daihan Glucose Injection (5%) 50 ml, 100 ml, 200 ml, 500 ml, 1000 ml

Dextrose 5 g per 100 ml

Daihan Glucose Injection (10%) 500 ml, 1000 ml

Dextrose 5 g per 100 ml

Daihan Glucose Injection (20%)

20 ml/A, Dextrose 20 g per 100 ml

1)-9 Gluorange 100 Solution (Korea McNulty's Co., Ltd.)

1 ml/A, Glucose 0.338 g per 1 ml

1)-10 Jeil Dextrose Injection (Je Il Pharmaceutical Co., Ltd.)

20 ml/A, Dextrose 20 g per 100 ml

1)-11 CJ Dextrose Injection 5% 50 ml, 100 ml, 200 ml, 500 ml, 1000 ml

Dextrose 5 g per 100 ml

1)-12 Choongwae 5% 50 ml, 200 ml

Dextrose 5 g per 100 ml

Further, amino acid may include one selected from the following products.

An injection, as a colorless or creamy yellow and transparent chemical liquid, with a rubber plug for infusion

1)-1 Clinimix 85 Injection 1 L, 1.5 L (Ilyang Pharmaceutical Co., Ltd.)

Amino acids 8.5% (Solution A 1 L) Dextrose 330 g (Solution B 1 L)

1)-2 Choongwae Trophamine Injection 6% 100 ml, 200 ml (Choongwae Pharma, Co., Ltd.)

1)-3 Choongwae Hepatamin Injection 250 ml (Choongwae Pharma, Co., Ltd.)

1)-4 Cafsol Injection 250 ml, 500 ml (Chong Kun Dang Pharmaceutical Corp.)

1)-5 Yungjin Intrafusin 10% Injection 250 ml, 500 ml (Yungjin Pharm. Co., Ltd.)

1)-6 Proamin Injection 10% (Hanall Biopharma)

1)-7 Hepavia Injection 250 ml, 500 ml (Dai Han Pharm. Co., Ltd.)

1)-8 Liversol Injection 500 ml (Shinpoong Co., Ltd.)

1)-9 Hanall Morihepamin Injection (Hanall Biopharma)

1)-10 Nephrisol 5.6% (Dai Han Pharm. Co., Ltd.)

1)-11 Glamin Injection 250 ml, 500 ml (Fresenius Kabi Korea)

1)-12 Primene 10% (Baxter)

1)-13 Doctorlamine Injection (Choongwae Pharma, Co., Ltd.)

1)-14 Aminoglu Injection 250 ml, 500 ml, 1000 ml (MG Medi Green)

1)-15 Aminoven Infant 6% Injection (Fresenius Kabi Korea)

1)-16 Aminosteril-KE Injection 10% (Dai Han Pharm. Co., Ltd.)

1)-17 Aminofusin-TPN Injection (Yungjin Pharm. Co., Ltd.)

1)-18 Combiflex MCT Plus Injection 1250 ml, 1875 ml (Choongwae Pharma, Co., Ltd.)

1)-19 Combiflex MCT Peri Injection 1250 ml, 1875 ml (Choongwae Pharma, Co., Ltd.)

1)-20 Combiflex MCT Special Injection 1250 ml (Choongwae Pharma, Co., Ltd.)

1)-21 Saeronamin Injection 250 ml (Dai Han Pharm. Co., Ltd.)

Furthermore, a vitamin supply source may be selected from the following products.

Orange, scentless freeze-dried powder in a brown glass vial

1)-1 Tamipool inj. (Celltrion Pharma. Inc.)

1)-2 Cetnevit inj. (Baxter)

Also, an antibiotic-antimycotic product may be selected from the following products.

1) Amphotericin B

Fungizone Injection 50 mg (BMS Pharmaceutical Korea Ltd.)

Amphotericin B 50 mg

2) Streptomycin Sulfate

Chongkundang Streptomycin Sulfate Injection (Chong Kun Dang Pharmaceutical Corp.)

Streptomycin Sulfate 1 g

3) Penicillin G Potassium

Kunwha Potassium Penicillin-G Injection 5 million U (Kunwha Pharmaceutical Co., Ltd.)

Further, as an osmoticum, one of the following components may be selected.

1) Albumin

An injection of a colorless or pale yellow and transparent solution in a colorless and transparent vial

1)-1 Greencross Human Serum Albumin Injection 5% 100 ml, 250 ml (Green Cross Corp.)

Human Serum Albumin 5 g per 100 ml

Greencross Human Serum Albumin Injection 20% 50 ml, 100 ml (Green Cross Corp.)

Human Serum Albumin 20 g per 100 ml

1)-2 SK Albumin Injection 5% 250 ml (SK Chemicals)

Human Serum Albumin 5 g per 100 ml

SK Albumin Injection 20% 50 ml, 100 ml

Human Serum Albumin 20 g per 100 ml

2) Dextran (Colloidal Solution)

A colorless bottle filled with colorless contents having slight sweetness

2)-1 Hemadex-D Injection (Dextran-40 Injection) (Samsung Pharm. Ind. Co., Ltd.)

Dextran N/A Glucose N/A

2)-2 CJ Dextran-40D Injection (CJ Cheiljedang)

Dextran 40 50 g and Dextrose 25 g per 500 ml

In connection with the present invention, examples of an infusion or injection preparation for vitamin supply; an infusion or injection preparation for amino acid supply; and an infusion or injection preparation for inorganic salt supply which can be used in U.S., Japan, and other countries as well as Korea are described in FIGS. 14 to 15 and FIGS. 16 to 17.

In the present invention, the term “complete medium” refers to a medium which can actually culture cells. For example, complete medium=commercial culture medium+10% serum.

Hereinafter, the present invention will be explained.

The inventors of the present invention have developed cell therapy products for regeneration/repair of cartilage so far, and based on the findings obtained thereby, the inventors of the present invention came to think that various cell resources for treatment including cell therapy products undergo in vitro culture for early clinical process of regenerative medicine and it is necessary to prepare a safe medium with infusion preparations and injection preparations already approved as medicines without using commercial medium most of which contain toxic substances or the like.

Generally, in order to maintain or multiply normal cells and cell lines in an apparatus for culture such as a culture plate or the like, an appropriately selected complete culture medium or serum-free culture medium is used. The complete culture medium usually refers to a mixture of a commercial serum-free culture medium such as MEM (minimum essential medium), DMEM (Dulbecco's modified eagle medium), F-12K medium, RPMI 1640 medium, and McCoy's 5A medium (GIBCO, Invitrogen Cell Culture Products, 2002 Catalog, Invitrogen, NY; BioWhittaker Cell Biology Products, 2002-2003 Catalog, Cambrex, Md.) with serum of about 10% with respect to the total volume. As serum, there has been mainly used fetal bovine serum (known as FBS) separated from a cow. Serum comprises various components known or unknown as being highly involved in cell proliferation and thus is almost essential for cell culture. Therefore, even if cells can be maintained or multiplied in a serum-free culture medium through compulsory adaptation in a culture apparatus (Berg et al. High-level expression of secreted proteins from cells adapted to serum-free suspension culture. Biotechniques 14, 972-978 (1993); Haldankar et al. Stable production of a human growth hormone antagonist from CHO cells adapted to serum-free suspension culture. Biotechnol. Prog. 15, 336-346 (1999); Sinacore et al. Adaptation of mammalian cells to growth in serum-free media. Mol. Biotechnol. 15, 249-257 (2000)), it is more general to use a complete culture medium for cell culture.

Further, in the present invention, serum is prepared from cord blood and autologous blood discarded as medical waste to obtain a raw material of heterogeneous animal-derived xeno-free serum without using Fetal Bovine Serum (FBS), and a complete medium based on an infusion preparation is developed.

Regarding a necessity for preparation of a xeno-free culture medium, when a culture is carried out for the purpose of cell therapy, it is desirable to minimize mixing of heteromaterials considering dangers of mad cow disease or the like and future clinical test approval.

Currently, FBS (fetal bovine serum) has been often used, but in the present invention, it is necessary to develop human-derived serum resources (autologous blood and cord blood of a patient) which can be substituted for FBS.

Accordingly, in the present invention, a complete medium is developed using autologous blood and cord blood-derived serum as an alternative to FBS.

For reference, commercial culture media for cell culture are described in detail at http://www.invitrogen.com/site/us/en/home/Products-and-Services/Applications/Cell-Culture/Mammalian-Cell-Culture/Classical Media.html, and may include DMEM, DMEM/F-12, F-10, Ham's F-12, MEM, RPMI medium 1640, and the like. A composition of DMEM (high glucose) as a representative culture medium is described at http://www.invitrogen.com/site/us/en/home/support/Prouct-Technical-Resources/media_formulation.10.html, as listed in the following Table 1.

TABLE 1

| | | | |
|---|---|---|---|
| | Molecular | Concentration | |
| Composition | Weight | (mg/L) | mM |

Amino Acids

| | | | |
|---|---|---|---|
| Glycine | 75 | 30 | 0.4 |
| L-Arginine | 211 | 84 | 0.398 |
| hydrochloride | | | |
| L-Cystine 2HCl | 313 | 63 | 0.201 |
| L-Glutamine | 146 | 584 | 4 |
| L-Histidine | 210 | 42 | 0.2 |
| hydrochloride-H₂O | | | |
| L-Isoleucine | 131 | 105 | 0.802 |
| L-Leucine | 131 | 105 | 0.802 |
| L-Lysine hydrochloride | 183 | 146 | 0.798 |
| L-Methionine | 149 | 30 | 0.201 |
| L-Phenylalanine | 165 | 66 | 0.4 |
| L-Serine | 105 | 42 | 0.4 |
| L-Threonine | 119 | 95 | 0.798 |
| L-Tryptophan | 204 | 16 | 0.0784 |
| L-Tyrosine disodium | 261 | 104 | 0.398 |
| salt dihydrate | | | |
| L-Valine | 117 | 94 | 0.803 |

Vitamins

| | | | |
|---|---|---|---|
| Choline chloride | 140 | 4 | 0.0286 |
| D-Calcium pantothenate | 477 | 4 | 0.00839 |
| Folic Acid | 441 | 4 | 0.00907 |
| Niacinamide | 122 | 4 | 0.0328 |
| Pyridoxine | 204 | 4 | 0.0196 |
| hydrochloride | | | |
| Riboflavin | 376 | 0.4 | 0.00106 |
| Thiamine hydrochloride | 337 | 4 | 0.0119 |
| i-Inositol | 180 | 7.2 | 0.04 |

Inorganic Salts

| | | | |
|---|---|---|---|
| Calcium Chloride | 111 | 200 | 1.8 |
| (CaCl₂) (anhyd.) | | | |
| Ferric Nitrate | 404 | 0.1 | 0.000248 |
| (Fe(NO₃)₃″9H₂O) | | | |
| Magnesium Sulfate | 120 | 97.67 | 0.814 |
| (MgSO₄) (anhyd.) | | | |
| Potassium Chloride | 75 | 400 | 5.33 |
| (KCl) | | | |
| Sodium Bicarbonate | 84 | 3700 | 44.05 |
| (NaHCO₃) | | | |
| Sodium Chloride (NaCl) | 58 | 4750 | 81.9 |
| Sodium Phosphate | 138 | 125 | 0.906 |
| monobasic | | | |
| (NaH2PO4—H₂O) | | | |

Other Components

| | | | |
|---|---|---|---|
| D-Glucose (Dextrose) | 180 | 4500 | 25 |
| HEPES | 238 | 5958 | 25.03 |
| Phenol Red | 376.4 | 15 | 0.0399 |

The inventors of the present invention reviewed infusion preparations and injection preparations based on the components of the DMEM (high glucose).

Firstly, as a vitamin supply source, Tamipool inj. (Celltrion Pharma. Inc.) is selected as a basic component since it satisfies 6 of 8 necessary vitamins for the DMEM and inositol and choline as the other two deficient vitamins and folic acid can be supplemented by separate infusion preparations.

Further, for vitamins, an alternative product is as follows:

(1) Cetnevit inj. (Baxter); satisfying 5 of the necessary vitamins and containing 7 minor components, which are not contained in the DMEM, more than 6 in Tamipool inj.

Furthermore, for supplement of vitamins, the following three products can be used independently or in combination:

(1) Lyvitol Solution (Cho-A Pharm. Co., Ltd.); supplementing 6000 mg of inositol, in which Tamipool inj. is deficient, per 100 ml.

(2) Folic Acid Injection 5 mg in 1 ml (phebra); easy to prepare in the form of an infusion.

(3) Bidoxin Injection (Pyridoxine hydro chloride) (Dai Han Pharm. Co., Ltd.); supplementing pyridoxine, in which Tamipool inj. is deficient, as an ampoule injection containing 50 mg of pyridoxine.

Also, an alternative product for supplement of vitamins is as follows:

(1) Wolthby Liq. (Dong Wha Pharm Co., Ltd.); supplementing 6000 mg of inositol, in which Tamipool inj. is deficient, per 100 ml.

containing 0.3 mg of cyanocobalamin, which is not contained in the DMEM, more than 3 g in the Lyvitol Solution.

Further, for supply of “amino acids”, Primene 10% (Baxter) is selected as a basic component since it contains most of the amino acids contained in the DMEM, as an amino acid infusion for baby and infant, and its necessary components are also similar in value to those of the DMEM.

Furthermore, for supply of “inorganic salts”, the following five products may be used alone or in combination as a basic component.

(1) Choongwae Hartman Solution (Choongwae Pharma, Co., Ltd.); not containing dextrose and minerals, which are not contained in the DMEM, having similar values of calcium, potassium, and sodium, and easily adjusted in osmotic pressure by separately supplementing magnesium and iron.

(2) Daihan Magnesium Sulfate Injection 10% (Dai Han Pharm. Co., Ltd.)

(3) Ferinject Injection (Choongwae Pharma, Co., Ltd.); high-concentration Fe injection containing 50 mg of Fe per ml

(4) Daihan Sodium Bicarbonate Injection 8.4% (Dai Han Pharm. Co., Ltd.); containing 1.68 mg of Sodium Bicarbonate per 20 ml in the form of an injection. Low price.

(5) Colclean Solution (Taejoon Pharm Co., Ltd.); only containing 180 mg of dibasic sodium phosphate and 480 mg of monobasic sodium phosphate per ml as components for adjusting a pH at an adequate ratio therebetween.

Further, as alternative products to these products, the following five products may be used alone or in combination.

(1) B.Braun Hartmanns Injection 500 ml (B. Braun Korea)

(2) Magnesin Injection 10% (Magnesium Sulfate Hydrate) (Daewon Pharm. Co., Ltd.)

(3) Venoferrum Injection (Choongwae Pharma, Co., Ltd.) containing 20 mg of Fe per ml.

(4) Daewon Sodium Bicarbonate Injection (Daewon Pharm. Co., Ltd.)

(5) Phospanol Solution (Oral Sodium Phosphate Solution) (Dongindang Pharmaceutical Co., Ltd.)

Further, serum is prepared from cord blood and autologous blood discarded as medical waste, and a complete medium based on the above infusion preparations is developed.

### Effect

As can be seen from the present invention, the present invention can be used as an alternative to commercial culture media since the present invention uses infusion preparations and injection preparations already approved as medicines, and, thus, there is no difference in shape and growth morphology of cell between the commercial culture media and the medium of the present invention.

### DESCRIPTION OF DRAWINGS

FIG. 1 shows infusion or injection preparations having compositions of cell culture media in various combinations used in the present invention, including an infusion or injection preparation for amino acid supply, an infusion or injection preparation for vitamin supply, an infusion or injection preparation for inorganic salt supply, an infusion or injection preparation for glucose supply, other supplement infusion or injection preparations, and an antibiotic and antimycotic infusion or injection preparation.

FIG. 2 and FIG. 3 are tables listing a composition of FCM-high glucose (FCM-HG) prepared with an infusion or injection preparation for amino acid supply, an infusion or injection preparation for vitamin supply, an infusion or injection preparation for inorganic salt supply, an infusion or injection preparation for glucose supply, other supplement infusion or injection preparations, and an antibiotic and antimycotic infusion or injection preparation measured to be similar to DMEM-high glucose (DMEM-HG) (10313, Gibco) in components and amounts thereof.

FIG. 4 provides photos of the infusion or injection preparation for amino acid supply, the infusion or injection preparation for vitamin supply, the infusion or injection preparation for inorganic salt supply, an infusion or injection preparation for glucose supply, the other supplement infusion or injection preparations, and the antibiotic and antimycotic infusion or injection preparation used in FIG. 2 and FIG. 3;

FIG. 5 provides a photo of cell culture medium prepared by combining an infusion or injection preparation for amino acid supply, an infusion or injection preparation for vitamin supply, an infusion or injection preparation for inorganic salt supply, an infusion or injection preparation for glucose supply, other supplement infusion or injection preparations, and an antibiotic and antimycotic infusion or injection preparation, and specifically by mixing 5 ml Primene 10% infu. (Baxter, Ill., USA), 1.25 ml Tamipool inj. (Celltrion, Seoul, Korea), 500 ml Hartmann's soln. infu. (Dai Han Pharm. Co., Ltd., Seoul, Korea), 485 μl magnesium sulfate 10% inj. (Dai Han Pharm. Co., Ltd., Seoul, Korea), 1 μl Ferinject inj. (Choongwae Pharma, Co., Ltd., Seoul, Korea), 22 ml sodium bicarbonate 8.4% inj. (Huons Co., Ltd, Gyung gi, Korea), 100 μl Colclean inj. (Taejoon Pharm Co., Ltd, Seoul, Korea), 4.5 ml 50% dextrose inj. (Choongwae Pharm., Co., LTD., Seoul, Korea), 100,000 unit/L penicillin G potassium inj. (Kunwha Pharmaceutical Co., Ltd., Seoul, Korea), 100 mg/L streptomycin sulfate inj. (Chong Kun Dang Pharmaceutical Corp., Seoul, Korea), 0.25 mg/L amphotericine B of Fungizone inj. (BMS Pharmaceutical Korea Ltd., Seoul, Korea), and 10% FBS (Gibco) and filtering the mixture with a 0.22 μm filter system (Corning Inc., NY, USA).

FIG. 6 provides a photo on the left showing cell culture using a cell culture medium of the present invention prepared by combining an infusion or injection preparation for amino acid supply, an infusion or injection preparation for vitamin supply, an infusion or injection preparation for inorganic salt supply, an infusion or injection preparation for glucose supply, other supplement infusion or injection preparations, and an antibiotic and antimycotic infusion or injection preparation, and a photo on the right showing cell culture using a commercial DMEM-HG.

FIG. 7 provides photos of human fat synovium-derived mesenchymal stem cells (FS-MSCs), and specifically, FIGS. 7A, 7B, and 7C show Human Fat Synovium cells cultured using a commercial DMEG-HG (respectively, 24 hours, 48 hours, and 72 hours after culture from the left) and FIGS. 7D, 7E, and 7F are photos of Human Fat Synovium cells cultured using a FCM-HG of the present invention (respectively, 24 hours, 48 hours, and 72 hours after culture from the left). It can be seen from this result that the commercial culture medium and the culture medium of the present invention are not much different from each other in shape and growth morphology of cell and are similar to each other without any particular opinion.

FIG. 8 is a graph illustrating counted numbers of the cells in FIGS. 7A, 7B, 7C, 7D, 7E, and 7F. The Human Fat Synovium cells respectively cultured using the DMEM-HG and the FCM-HG are multiplied as time passes (24 hours, 48 hours, and 72 hours). Although the number of the Human Fat Synovium cells cultured using the DMEM-HG is slightly higher than the number of the Human Fat Synovium cells cultured using the FCM-HG, a statistically significant difference (P<0.05) cannot be seen. Cell Counter: AccuChip kit AdamMCcellcounter (NanoEnTek Inc., Seoul, Korea)

FIG. 9 is a graph illustrating a pH of the DMEG-HG and the FCM-HG each culturing the Human Fat Synovium cells for 24 hours, 48 hours, and 72 hours, and the DMEG-HG and the FCM-HG each culturing the Human Fat Synovium cells exhibit the same morphology where a pH tends to gradually increase as time passes (0 hour, 24 hours, 48 hours, and 72 hours). A statistically significant difference (P<0.05) cannot be seen between the DMEM-HG group and the FCM-HG group. pH Meter: Mettler ToledoSevenEasy pH meter

FIG. 10 is a graph illustrating an osmotic pressure of the DMEG-HG and the FCM-HG each culturing the Human Fat Synovium cells for 24 hours, 48 hours, and 72 hours, and the DMEG-HG and the FCM-HG each culturing the Human Fat Synovium cells exhibit the same morphology where an osmotic pressure tends to gradually increase as time passes (culture for 0 hour, 24 hours, 48 hours, and 72 hours). A statistically significant difference (P<0.05) cannot be seen between the DMEM-HG group and the FCM-HG group. Osmometer: ‘FiskeMicro-Osmometer (Model 210) (FiskeAssociates Inc., Mass., USA).

FIG. 11 provides photos of a rat heart myocardium-derived H9c2 (2-1) cell line (ATCC, VA, USA), and specifically, FIGS. 11A, 11B, and 11C show a Rat H9c2 cell line cultured using the DMEG-HG (respectively, 24 hours, 48 hours, and 72 hours after culture from the left) and FIGS. 11D, 11E, and 11F show a Rat H9c2 cell line cultured using the FCM-HG (respectively, 24 hours, 48 hours, and 72 hours after culture from the left). It can be seen from this result that the commercial culture medium and the culture medium of the present invention are not much different from each other in shape and growth morphology of cell and are similar to each other without any particular opinion.

FIG. 12 is a photo showing FCM-HG conditioned media, which are divided by 1 ml into 1.5 ml tubes and freeze-dried, as used for culture of the present invention, and FIG. 13 shows powder of the FCM-HG conditioned media collected into a 50 ml tube from 20 freeze-dried powder tubes of FIG. 12 as a substance in powder form obtained by freeze-drying the conditioned media. This substance is expected to be used for treatment, regeneration, health improvement through a transfer into a body including injection.

FIG. 14 and FIG. 15 are lists of infusion or injection preparations for vitamin supply, infusion or injection preparations for amino acid supply, infusion or injection preparations for inorganic salt supply, infusion or injection preparations for glucose supply, inorganic salt supplements, and antibiotic and antimycotic infusion or injection preparations which can be used in the U.S.A.

FIG. 16 and FIG. 17 are lists of infusion or injection preparations for vitamin supply, infusion or injection preparations for amino acid supply, infusion or injection preparations for inorganic salt supply, infusion or injection preparations for glucose supply, inorganic salt supplements, and antibiotic and antimycotic infusion or injection preparations which can be used in Japan.

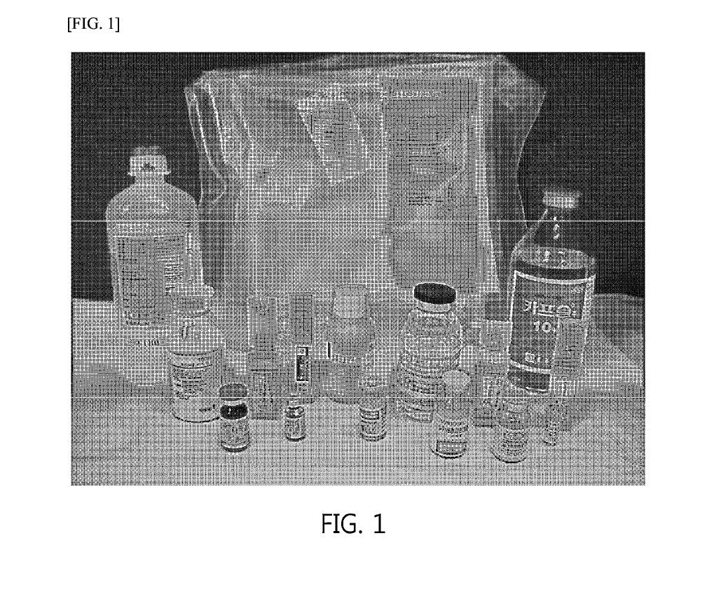

### BEST MODE

Hereinafter, the present invention will be explained in more detail with reference to non-limiting examples. However, the following examples are described for illustrating the present invention, but should not be construed as limiting the scope of the present invention.

### Example

### Preparation of Fluid-Based Culture Medium (FCM)

In order to prepare a fluid-based culture medium (FCM) of the present invention, B.Braun Hartmanns Injections 500 ml and 1000 ml (B. Braun Korea) were used as a basic solution for supply of inorganic salts, and these products contain Calcium Chloride 0.027 g, Potassium 0.04 g, Sodium Chloride 0.6 g, and Sodium Lactate 0.312 g per 100 ml.

Further, as a supply source of MgSO₄ anhyd., Daihan Magnesium Sulfate Injection 10% (Dai Han Pharm. Co., Ltd.) was used, and this product contains Magnesium Sulfate 2 g per 20 ml/A.

Furthermore, as a supply source of Fe(NO₃)₃.9H₂O, Ferinject Injections 2 ml and 10 ml were used, and these products contain ferric hydroxide carboxymaltose complex 180 mg (50 mg as Fe) per 1 ml.

Moreover, as a supply source of NaHCO₃, Huons Sodium Bicarbonate Injection 8.4% (Huons Co., Ltd.) was used, and this product contains 20 ml/A and Sodium Bicarbonate 1.68 g.

Also, as a supply source of NaH₂PO₄ and Na₂HPO₄, Colclean Solution (Taejoon Pharm Co., Ltd.) was used, and this product contains 1 ml/A, Dibasic sodium phosphate 180 mg, and Monobasic sodium phosphate 480 mg.

Further, as a supply source of glucose, Choongwae Dextrose Injection 50% 100 ml was used, and this product contains Dextrose 50 g per 100 ml.

Furthermore, as a supply source of amino acid, Primene 10% (Baxter) was used, and this product contains Aminoacetic Acid (Glycine) 4.00 g, L-alanine 8.00 g, L-arginine 8.40 g, L-aspartic acid 6.00 g, L-cysteine 1.89 g, L-glutamic acid 10.00 g, L-histidine 3.80 g, L-leucine 10.00 g, L-Isoleucine 6.70 g, L-lysine 22.00 g (11.00 g as L-lysine), L-methionine 2.40 g, L-ornithine HCl 3.18 g, L-phenylalanine 4.20 g, L-proline 3.00 g, L-serine 4.00 g, L-threonine 3.70 g, L-tryptophan 2.00 g, L-tyrosine 0.45 g, L-valine 7.60 g, and Taurine 0.60 g per 1000 mL.

Moreover, as a supply source of vitamin, Tamipool inj. (Celltrion Pharma. Inc.) was used, and this product contains Ascorbic Acid 100 mg, Biotin 60 μg, Cyanocobalamin 5 μg, Dexpanthenol 15 mg, Ergocalciferol 200 I.U, Folic Acid 400 μg, Nicotinamide 40 mg, Pyridoxine HCl 4.86 mg, Riboflavin sodium phosphate 3.6 mg, Thiamine HCl 3.81 mg, Tocopherol Acetate 10 mg, and Vitamin A 3300 I.U, and for folic acid supplement, Foliamin Injection (Japanese product) was used.

Also, for supply of Amphotericin B as a supply source of antibiotic-antimycotic, Fungizone Injection 50 mg (BMS Pharmaceutical Korea Ltd.) was used, and this product contains Amphotericin B 50 mg, and for supply of Streptomycin Sulfate, Chongkundang Streptomycin Sulfate Injection (Chong Kun Dang Pharmaceutical Corp.) was used, and this product contains Streptomycin Sulfate 1 g. For supply of Penicillin G Potassium, Kunwha Potassium Penicillin-G Injection 5 million U (Kunwha Pharmaceutical Co., Ltd.) was used.

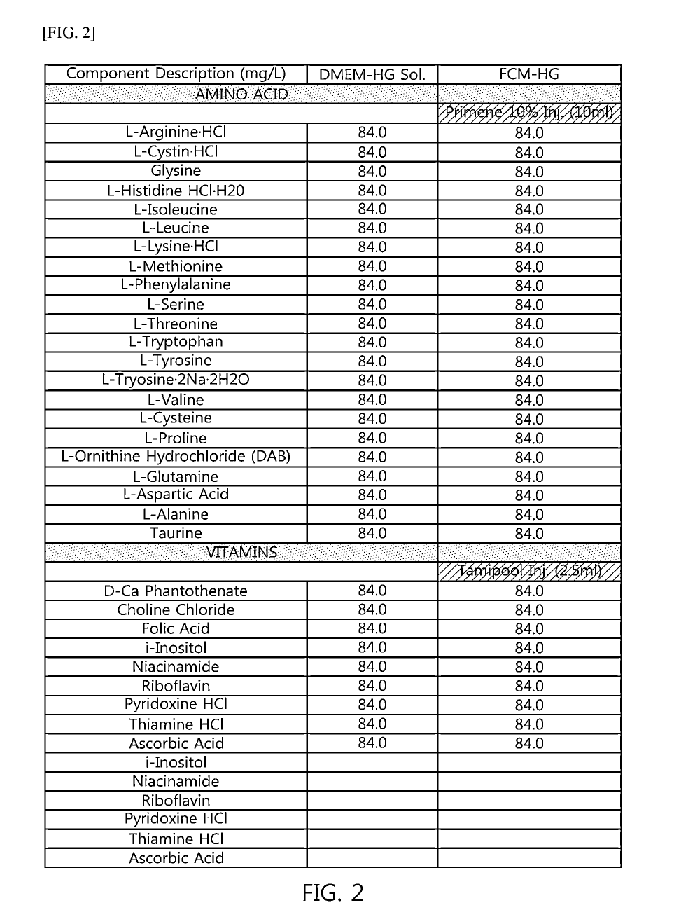

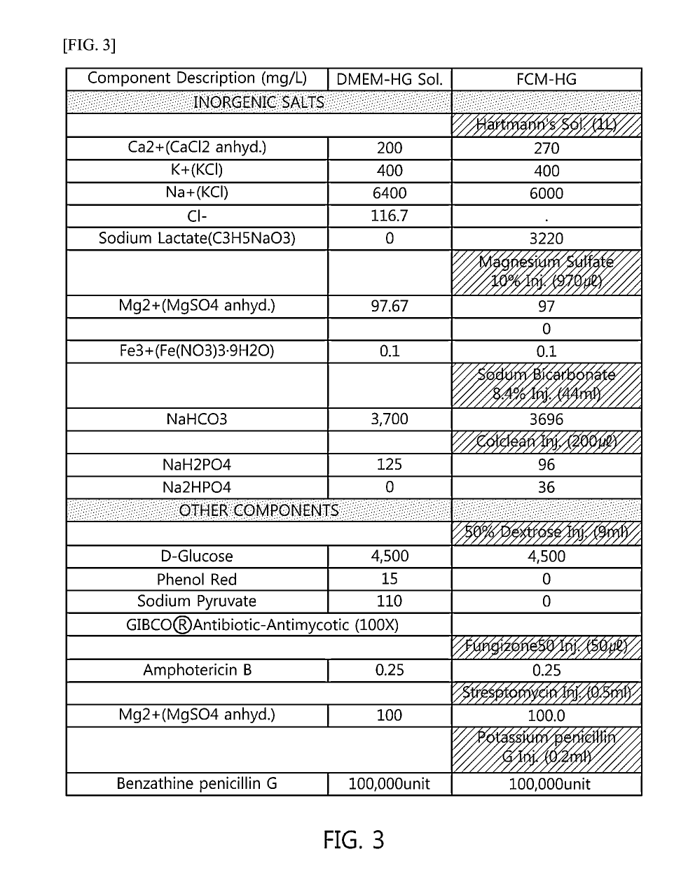

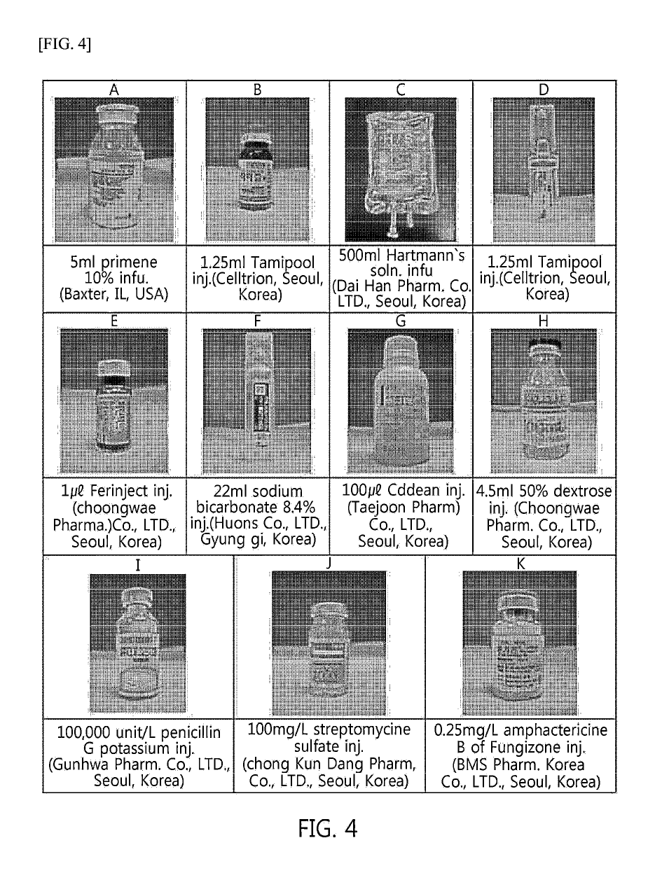

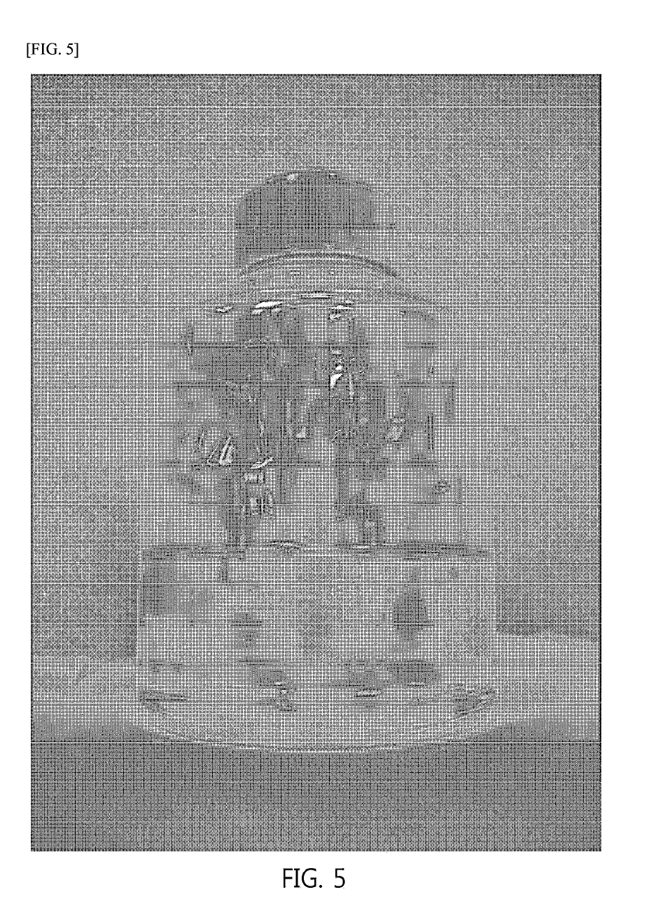

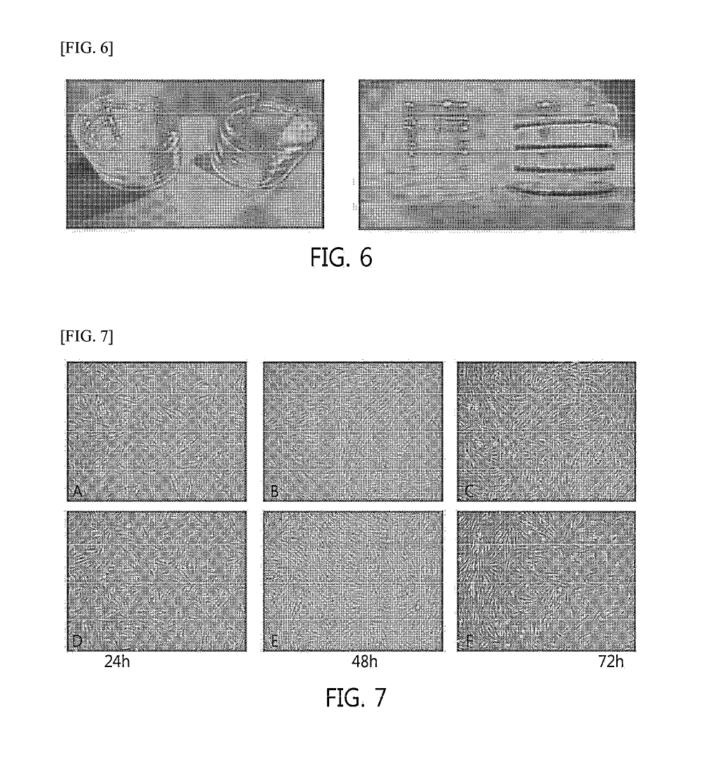

In the present invention, the above-described components were mixed at a volume ratio described in FIG. 2 and FIG. 3 so as to prepare a fluid-based culture medium (FCM). When the respective components of the injection or infusion preparations were mixed, preparation was made within a clean bench with mixing at room temperature while maintaining an aseptic condition. After mixing, the mixture was filtered with a corning filter system 500 ml.

### Experimental Example

### Result of Cell Culture

The culture medium of Example was a mixture of medicines including the identical and/or similar components to the DMEM-HG 10313 listed in the component analysis lists an on the right side of FIG. 2 and FIG. 3, and did not contain or was deficient in five necessary components of the DMEM in total, i.e. Sodium Pyruvate, L-CystinHCl, L-Tyrosine2Na₂H₂O, Choline Chloride, and i-Inositol. Phenol Red as an indicator was excluded. The Tamipool inj. used as a source of vitamin contained other minor components. With the present culture medium, five kinds of cells of human-derived and animal-derived cell lines, and commercial cell lines, i.e. RINm, EPC, PSY, FS-MSCs, and H9c2(2-1) were cultured, and after 12 hours, 24 hours, 48 hours, and 72 hours, the status of the cells were observed.

Of the above cell lines, RINm and H9c2(2-1) were purchased from the ATCC under Catalogue No. CRL-2057 (Rattus norvegicus (rat) Source: Organ—pancreas, Strain—NEDH, Tissue—islet cell tumor, Disease—insulinoma, Cell Type—beta cell) and Catalogue No. CRL-1446 (Rattus norvegicus (rat) Source: Organ—heart, Strain—BD1×, Tissue—myocardium), respectively. EPC is an endothelial progenitor cell obtained by in-vitro separation from human cord blood with reference to reference literature, and PSY as porcine synovium and FS-MSCs were obtained by in-vitro separation from swine synovial tissue and human synovial tissue using collagenase1 and culture with reference to reference literature (The development of 3-dimensional scaffold-free spheroid of endothelial progenitor cells on vascular regeneration, Tissue Engineering and Regenerative Medicine, 7:1, 42-48, 2010, Sang-Mo Kwon, Jae-Won Kim, and Jeong-Ik Lee; Transplantation of scaffold-free spheroids composed of synovium=derived cells and chondrocytes for the treatment of cartilage defects of the knee, European Cells & Materials Journal, 22, 275-290, 2011, Jeong Ik Lee, Masato Sato, Hyun Woo Kim, and Joji Mochida).

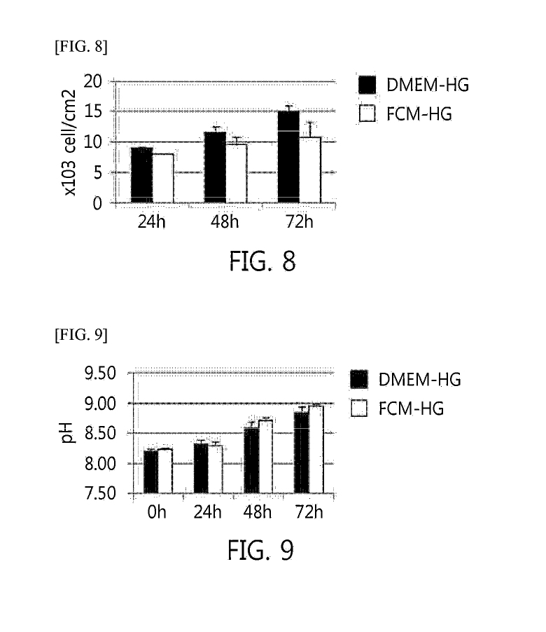

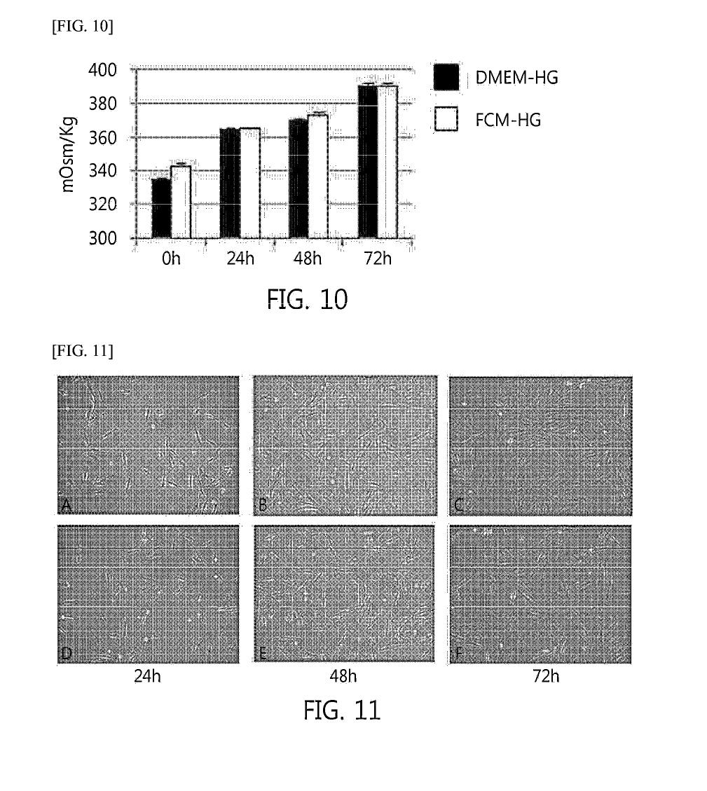

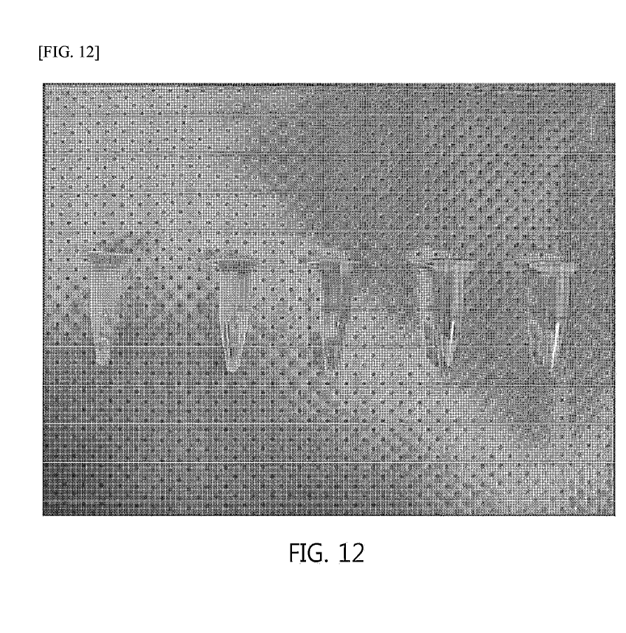

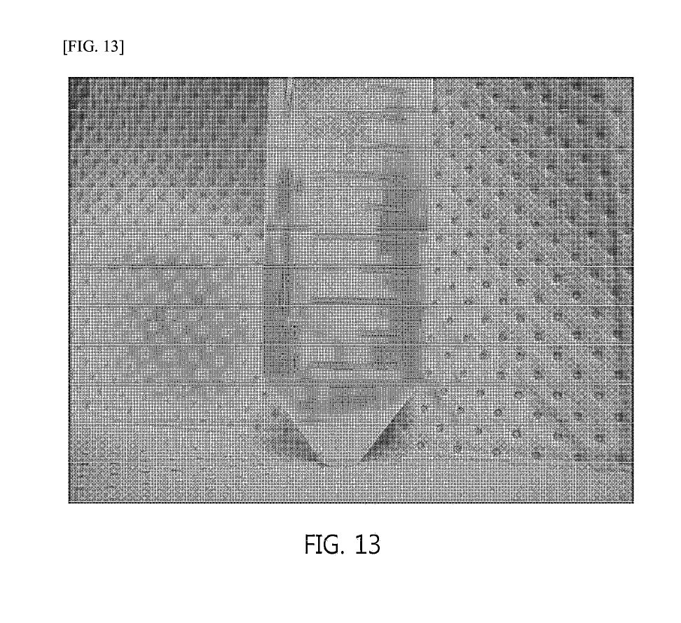

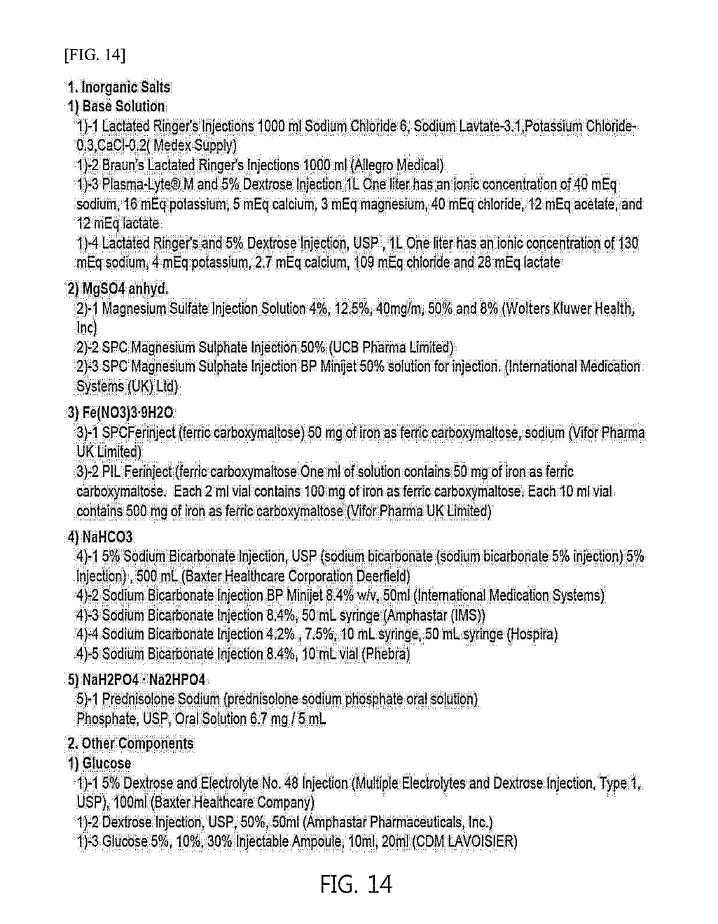

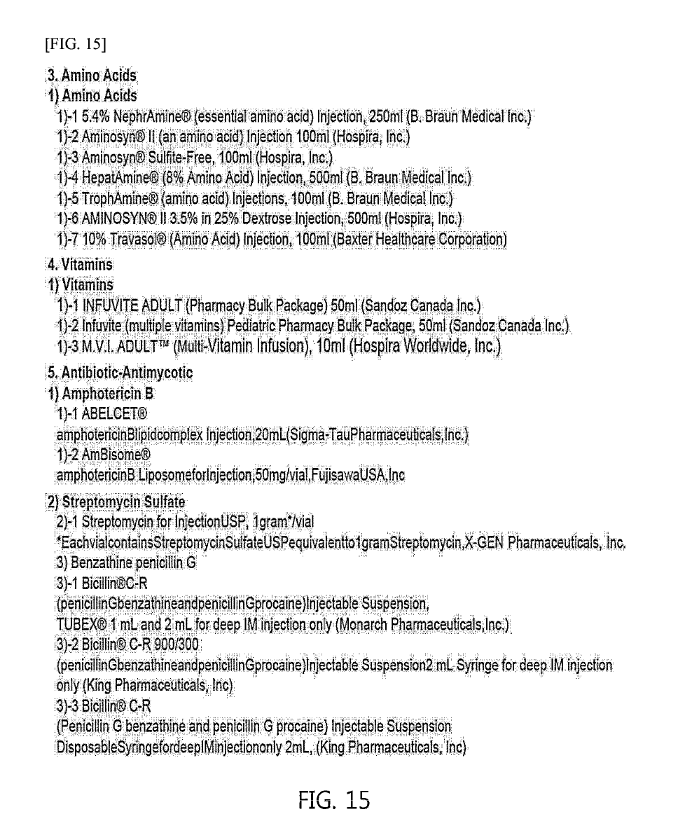

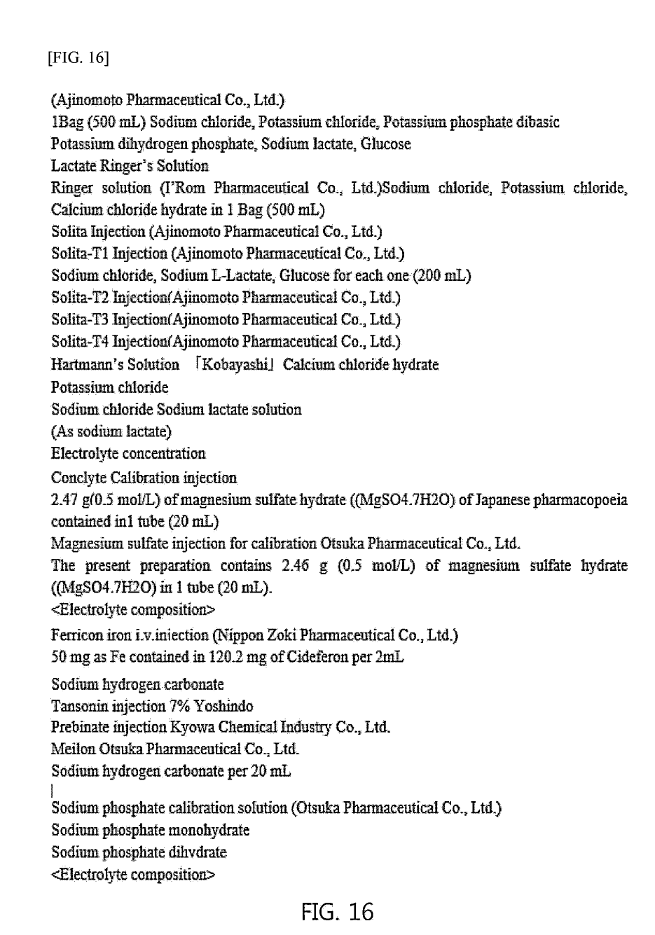

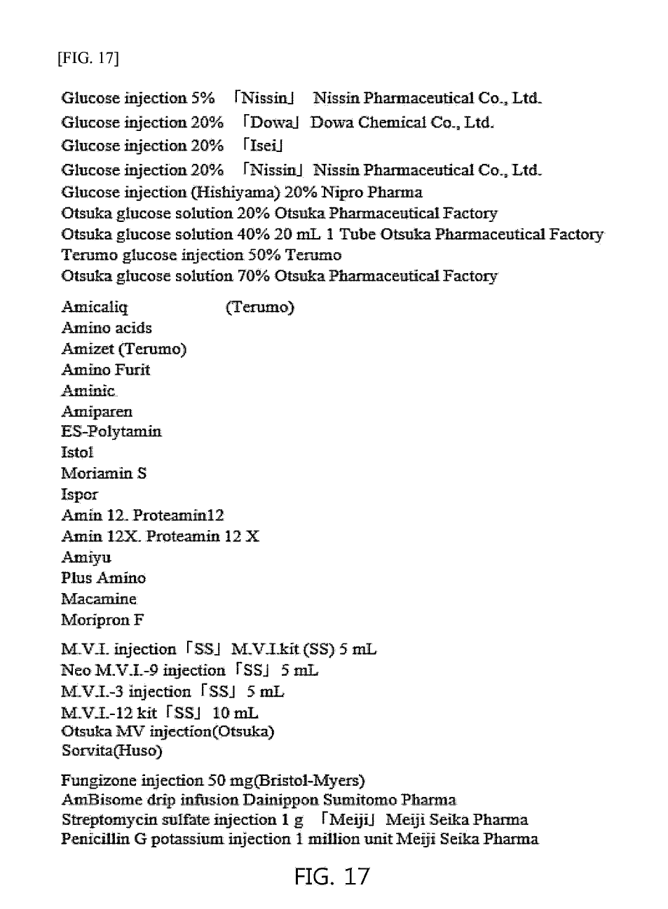

The results thereof were as shown in FIG. 7 to FIG. 11.

1. A preparation method of a cell culture medium, comprising: preparing a culture medium having a composition similar to a commercial cell culture medium by mixing
an infusion or injection preparation for vitamin supply,
an infusion or injection preparation for amino acid supply, and
an infusion or injection preparation for inorganic salt supply.

2. The preparation method of a cell culture medium of claim 1, wherein the infusion or injection preparation for vitamin supply is one or a combination selected from the group consisting of Tamipool inj. (Celltrion Pharma. Inc.), Cetnevit inj. (Baxter), INFUVITE ADULT (Sandoz Canada Inc.), Infuvite (Sandoz Canada Inc.), M.V.I. ADULT™ (Hospira Worldwide, Inc.), M.V.I. inj. (SS), M.V.I. kit, M.V.I.-9 inj. (SS), M.V.I.-3 inj. (SS), M.V.I.-12 kit (SS), Otsuka MV inj. (Otsuka), and Sorvita (Huso).

3. The preparation method of a cell culture medium of claim 1, wherein the infusion or injection preparation for amino acid supply is a preparation selected from the group consisting of Clinimix 85 inj. (Ilyang Pharmaceutical Co., Ltd.), Choongwae Tropamin Injection 6% (Choongwae Pharma, Co., Ltd.), Choongwae Hepatamine Injection (Choongwae Pharma, Co., Ltd.), Cafsol Injection (Chong Kun Dang Pharmaceutical Corp.), Yungjin Intrafusin 10% Injection (Yungjin Pharmaceutical Co., Ltd.), Proamin Injection 10% (Hanall Biopharma), Hepavia Injection (Dai Han Pharm. Co., Ltd.), Liversol Injection (Shin Poong Pharm. Co., Ltd.), Hanall Morihepamin Injection (Hanall Biopharma), Nephrisol 5.6% (Dai Han Pharm. Co., Ltd.), Glamin Injection (Fresenius Kabi Korea), Primene 10% (Baxter), Doctorlamine Injection (Choongwae Pharma, Co., Ltd.), Aminoglu Injection (MG Medi Green), Aminoven Infant 6% Injection (Fresenius Kabi Korea), Aminosteril-KE Injection 10% (Dai Han Pharm. Co., Ltd.), Aminofusin-TPN Injection (Yungjin Pharmaceutical Co., Ltd.), Combiflex MCT Plus Injection (Choongwae Pharma, Co., Ltd.), Combiflex MCT Peri Injection (Choongwae Pharma, Co., Ltd.), Combiflex MCT Special Injection (Choongwae Pharma, Co., Ltd.), Saeronamin Injection (Dai Han Pharm. Co., Ltd.), 5.4% NephrAmine Injection (B. Braun Medical Inc.), Aminosyn II Injection (Hospira, Inc.), Aminosyn Sulfite-Free (Hospira, Inc.), HepatAmine Injection (B. Braun Medical Inc.), TrophAmine Injection (B. Braun Medical Inc.), AMINOSYN II 3.5% in 25% Dextrose Injection (Hospira, Inc.), 10% Travasol Injection (Baxter Healthcare Corporation), Amicaliq (Terumo) Amizet XB (Terumo), Amino Furit, Aminic, amiparen, ES-Polytamin, Istr, Moriamin S, Ispor S 12%, Amin 12 Proteamin 12, Amin 12 X Proteamin 12 X, Amiyu, Plus Amino, Macamine, and Moripron F.

4. The preparation method of a cell culture medium of claim 1, wherein the infusion or injection preparation for inorganic salt supply is one or a combination selected from the group consisting of Daihan Hartmann Glucose Injection (Dai Han Pharm. Co., Ltd.), Hartmann Solution (Dai Han Pharm. Co., Ltd.), Hartmandex Solution (Choongwae Pharma, Co., Ltd.), Hartman Solution (Choongwae Pharma, Co., Ltd.), CJ Hartman Solution (CJ Cheiljedang), CJ Hartman-D Solution (CJ Cheiljedang), B.Braun Hartmanns Injection (B. Braun Korea), Daihan Isotonic Sodium Chloride Injection, Lactated Ringer's Injection (Medex Supply), Braun Lactated Ringer's Injection (Allegro Medical), Plasma-Lyte M and 5% Dextrose Injection, Lactated Ringer and 5% Dextrose Injection, EL-3 (Ajinomoto Pharmaceutical Co., Ltd.), Lactate Ringer's Solution, Ringer's Solution (I'Rom Pharmaceutical Co., Ltd.), Solita Injection (Ajinomoto Pharmaceutical Co., Ltd.), Solita-T1 Injection (Ajinomoto Pharmaceutical Co., Ltd.), Solita-T2 Injection (Ajinomoto Pharmaceutical Co., Ltd.), Solita-T3 Injection (Ajinomoto Pharmaceutical Co., Ltd.), Solita-T4 Injection (Ajinomoto Pharmaceutical Co., Ltd.), and Hartmann's Solution “Kobayashi” 500 mL calcium chloride dihydrate.

5. The preparation method of a cell culture medium of claim 1, further comprising:
adding glucose, an inorganic salt supplement, and an antibiotic and an antimycotic.

6. A cell culture medium composition comprising:
an infusion or injection preparation for vitamin supply;
an infusion or injection preparation for amino acid supply; and
an infusion or injection preparation for inorganic salt supply.

7. The cell culture medium composition of claim 6, further comprising:
glucose, an inorganic salt supplement, and an antibiotic and an antimycotic.

8. The cell culture medium composition of claim 6, wherein the composition is used as a general conservative solution or an immersion solution for cells and tissue for a short time or for a long time, or a portable solution for easy delivery.

9. The cell culture medium composition of claim 6, wherein the composition is used as a component of a preservation solution for preservation and delivery of an organ to be transplanted into a body.

10. The cell culture medium composition of claim 6, wherein the composition is used as an alternative to a buffer solution used in a molecular biological or biochemical experimental protocol.

11. A complete medium composition for cell therapy products, comprising:
the composition according to claim 6; and
serum derived from cord blood or autologous blood.

12-14. (canceled)
